(19) United States
(12) Patent Application Publication
(10) Pub. No.: US 2024/0375040 A1
(43) Pub. Date: Nov. 14, 2024

(54) Reactor System having Sorbent to Remove Carbon Dioxide

(71) Applicant: Saudi Arabian Oil Company, Dhahran (SA)

(72) Inventors: Mohammad Abdur Rakib, Dhahran (SA); Osamah Siddiqui, Dhahran (SA); Aadesh Harale, Dhahran (SA); Sai P. Katikaneni, Dhahran (SA); Salman Khaldi, Dammam (SA); Bandar Solami, Dammam (SA)

(21) Appl. No.: 18/314,491

(22) Filed: May 9, 2023

(51) Int. Cl. B01D 53/08; B01D 53/14; B01J 20/34

(52) U.S. Cl. CPC B01D 53/08; B01D 53/1475; B01J 20/3483; B01D 2257/504

(57) ABSTRACT

A system and method for removing, via a moving bed of sorbent, carbon dioxide from a reaction mixture in a reactor having a first region of a fixed bed of catalyst and a second region of the moving bed of sorbent separated from the first region by a perforated partition, performing in the reactor via the catalyst a reaction of reactants giving products of the reaction including at least a first product and carbon dioxide, sorbing carbon dioxide in the reactor by the sorbent to remove carbon dioxide from the reaction mixture having the reactants and the products, flowing sorbent having sorbed carbon dioxide from the reactor to a regenerator, heating the sorbent in the regenerator to remove sorbed carbon dioxide giving regenerated sorbent, flowing the regenerated sorbent to the reactor, and discharging an effluent including at least the first product from the reactor.

### TECHNICAL FIELD

This disclosure relates to catalytic reactions that generate carbon dioxide as byproduct.

### BACKGROUND

The conversion of a reversible chemical reaction is generally limited by thermodynamic equilibrium corresponding to the process conditions. Several techniques may be adopted in the industry to achieve conversions beyond equilibrium conversion. It may be desirable to increase conversion of reactants and favor generation of products of the chemical reaction. In other words, it may be beneficial to shift the reaction equation to the right toward product(s) of the reaction.

### SUMMARY

An aspect relates to a method of removing carbon dioxide from a reaction mixture in a reactor, including providing a feed having reactants to a reactor that is a catalytic reactor including a vessel, wherein an inside volume of the vessel has a first region having a fixed bed of catalyst and a second region having a moving bed of sorbent separated from the first region by a perforated partition. The method includes performing in the reactor via the catalyst a reaction of the reactants giving products of the reaction, the products including a first product and carbon dioxide. The method includes sorbing carbon dioxide in the reactor by the sorbent, thereby removing carbon dioxide from a reaction mixture having the reactants and the products in the reactor. The method includes moving sorbent having sorbed carbon dioxide from the reactor via lift gas through an outlet conduit to a sorbent regeneration vessel, heating the sorbent in the sorbent regeneration vessel to remove sorbed carbon dioxide giving regenerated sorbent having less sorbed carbon dioxide than the sorbent entering the sorbent regeneration vessel from the reactor, moving the regenerated sorbent from the sorbent regeneration vessel via lift gas through an inlet conduit to the reactor as sorbent introduced to the reactor, and discharging an effluent comprising at least one of the products from the reactor.

Another aspect relates to a method of removing carbon dioxide from a reaction mixture in a reactor, including providing a feed having reactants to a catalytic reactor including a reactor vessel, wherein an interior of the reactor vessel has a first region having a fixed bed of catalyst and a second region having a moving bed of sorbent, wherein the first region and the second region are separated by a perforated partition that restricts presence of the catalyst from the first region in the second region and restricts presence of sorbent from the second region in the first region. The method includes performing in the reactor vessel via the catalyst an equilibrium-limited reaction of the reactants giving products including carbon dioxide as a byproduct, and removing carbon dioxide from a reaction mixture including the reactants and the products in the reactor vessel by sorbing carbon dioxide from the reaction mixture onto the sorbent. The method includes discharging sorbent having sorbed carbon dioxide from the reactor vessel through an outlet conduit to a regeneration vessel, desorbing carbon dioxide from the sorbent in the regeneration vessel by heating the sorbent in the regeneration vessel, discharging sorbent from the regeneration vessel without the carbon dioxide desorbed from the sorbent through an inlet conduit to the reactor vessel, and discharging an effluent including the products from the reactor.

The details of one or more implementations are set forth in the accompanying drawings and the description below. Other features and advantages will be apparent from the description and drawings, and from the claims.

### BRIEF DESCRIPTION OF DRAWINGS

FIGS. 1, 2A, 2B, 2C, 3, and 4 are each a diagram of a reactor system having a catalytic reactor configured to perform an equilibrium-limited reaction that generates carbon dioxide (CO₂) as a product that is a byproduct.

FIGS. 5 and 6 are each a diagram of a catalytic reactor having internal guide plates.

FIG. 7 is a block flow diagram of a method of removing carbon dioxide from a reaction mixture in a reactor.

Like reference numbers and designations in the various drawings indicate like elements.

### DETAILED DESCRIPTION

Some aspects of the present disclosure are directed to a catalytic reactor system having sorbent (e.g., a moving bed of sorbent) to remove (e.g., sorb) carbon dioxide from a reaction mixture in the catalytic reactor. Thus, the technique may include removing carbon dioxide from the reaction mixture in the catalytic reactor, such as via sorbent (e.g., moving bed) in the reactor system. Aspects include a sorption-enhanced reactor configuration for equilibrium-limited reactions that produce carbon dioxide as byproduct.

Conversion of equilibrium-limited reactions can be enhanced by selective removal of at least one of the products, thereby forcing the system to move or shift towards a new equilibrium. The conversion of reversible reactions is generally limited by thermodynamic equilibrium corresponding to the process conditions. Techniques may be applied to achieve conversions beyond equilibrium conversion. For instance, unconverted reactants may be separated from the reactor product effluent and recycled to the reactor, such as to the feed stream that enters the reactor. Another practice, as indicated, involves in-situ removal of at least one of the products, so that the reaction further progresses in the forward direction to achieve a new equilibrium. The employment of sorbent to sorb byproduct CO₂ in the reactor implements this latter practice.

Embodiments of the present techniques are directed to reactors (catalytic reactors) having a fixed bed of catalyst for equilibrium-limited reactions (e.g., steam methane reforming, water gas shift, dimethyl ether synthesis, and so on) that produce carbon dioxide (CO₂) as byproduct. In operation, the reactor (catalytic reactor) may include a moving bed of sorbent to sorb (e.g., adsorb) CO₂. Therefore, the reactor may provide for continuous in-situ removal of the product CO₂ utilizing sorbent particles and thus enhancing the reaction conversion. In implementations herein, the sorbent particles are not mixed with the catalyst. The reaction mixture (products and unconverted reactants) inclusive of CO₂ generated in the reactor vessel moves from the catalyst region in the reactor vessel through a perforated partition to the sorbent bed in the reactor vessel. Beneficially, the catalyst is not subjected to the harsh conditions of regenerating the sorbent because regenerating the sorbent is performed in a regenerator vessel external from the reactor. The sorbent bed may be a moving bed of the sorbent particles that moves (e.g., continuously or in a continuous operation) between the reactor and the regenerator vessel. In implementations, the equilibrium-limited reaction may be characterized as “sorption-enhanced” in that sorbent sorbs in-situ (in the reactor) the byproduct CO₂ driving the in-situ reaction (in the reactor) toward product formation. Therefore, conversions beyond the standard equilibrium conversion may be achieved in shifting the reaction equation to the right toward products of the reaction equation.

Indeed, for reactions producing CO₂ as a byproduct, the sorption enhancement as discussed herein may be implemented. This can involve in-situ removal of CO₂ utilizing sorbent. The sorbent can be regenerated for further sorption, thereby providing for several cycles of sorption and regeneration, and thus enhancing (e.g., continuously) the reaction conversion as a result. Examples of CO₂-selective sorbents include limestone, dolomite, hydrotalcite-based or lithium zirconate-based sorbents, and so forth.

Embodiments of the present techniques provide a catalytic reactor configuration that enhances (advances, increases) conversion of reactions where CO₂ is a byproduct. An applicable equilibrium-limited reaction that can be a sorption-enhanced reaction with removal of CO₂ from the reaction mixture is steam methane reforming. Other equilibrium-limited reactions applicable for sorption enhancement that is removal of CO₂ from the reaction mixture via sorbent include the water-gas shift reaction and dimethyl ether (DME) (CH₃OCH₃) synthesis reaction, as listed below. Embodiments of the present techniques can be applied for other equilibrium-limited reactions producing CO₂ than those listed below. The ΔH₂₉₈⁰ given below is the heat of reaction at standard conditions of 298K and 1 atmosphere absolute, and is given in kilojoules per mole (KJ/mol). The second reaction listed under steam reforming is the water-gas shift reaction occurring in the steam reformer reactor via the reforming catalyst.

Steam reforming of hydrocarbons

CH₄+H₂O [symbol] 3H₂+CO ΔH₂₉₈⁰=206 kJ/mol

H₂O+CO [symbol] H₂+CO₂ ΔH₂₉₈⁰=−41 kJ/mol

CH₄+2H₂O [symbol] 4H₂+CO₂ ΔH₂₉₈⁰=165 kJ/mol

Water gas shift reaction

H₂O+CO [symbol] H₂+CO₂ ΔH₂₉₈⁰=−41 kJ/mol

Production of Dimethyl Ether (CH₃OCH₃)

3CO+3H₂ [symbol] CH₃OCH₃+CO₂ ΔH₂₉₈⁰=247 kJ/mol

The steam reforming may be steam reforming of methane (steam methane reforming) to give the products hydrogen (H₂), carbon monoxide (CO), and CO₂. As noted, the water-gas shift reaction may occur in the steam methane reformer (reactor). The feed to the steam methane reformer (reactor) may be the reactant methane and the reactant steam. In implementations, the methane can be fed as the primary component of natural gas fed to the reactor. Hydrocarbons other than methane can also be fed to the reactor as a steam methane reformer. In the reactor, the hydrocarbons other than methane may be reformed (or pre-reformed) into methane that is reformed into the products. The reactor effluent discharged from the reactor may have the products. In implementations, the steam methane reformer (reactor) effluent having the products can be characterized as synthesis gas (syngas) (H₂ and CO) having CO₂ and any unreacted methane and unreacted steam. The steam methane reforming reaction may be endothermic, as noted above. Therefore, heat may be applied to the reactor, such as with external electrical heaters or other types of heating. The catalyst may be a steam reforming catalyst that can be, for example, nickel catalyst, nickel-based catalyst, nickel-alumina catalyst, iron oxide catalyst, iron-chromium oxide catalyst or iron-chromium (Fe—Cr) catalyst, copper-zinc oxide catalyst or copper-zinc (Cu—Zn) catalyst, or any combinations thereof. Other catalysts are applicable.

For embodiments of the reactor as a reactor to perform the water gas shift reaction, the reactor may receive feed including the reactant CO and the reactant H₂O (water vapor or steam), and discharge a reactor effluent having the product H₂ and the product CO₂. The water-gas shift reaction may be slightly exothermic, as noted above. Heat can be removed from the reactor, such as with the reactor vessel having a heat-transfer jacket for a cooling medium. Other heat-removal (cooling) configurations are applicable. The catalyst can be labeled as a shift catalyst and may be metal-based catalyst. The shift catalyst may be similar to reforming catalyst. The shift catalyst may be copper and zinc on alumina, a copper-zinc-aluminum catalyst, iron-chromium (Fe—Cr) catalyst, copper-zinc (Cu—Zn) catalyst, chromium or copper promoted iron-based catalyst, or cobalt-molybdenum catalyst, or any combinations thereof. Other catalysts are applicable.

For the reactor as a DME synthesis reactor, the reactor may receive feed including the reactant CO and the reactant H₂, and discharge a reactor effluent having the product DME and the product CO₂. The DME synthesis reaction is generally endothermic, as noted above. Therefore, in operation, heat may be added to the reactor. The DME synthesis reaction equation given above may be known as the direct synthesis of DME from syngas. For DME synthesis from CO and H₂ (or from syngas having CO and H₂), the reactions may involve the three reactions of (1) methanol synthesis from CO and H₂, (2) methanol dehydration to give DME, and (3) water gas shift to give the overall reaction DME synthesis reaction listed above. The catalyst may be a bivalent catalyst or hybrid catalyst to facilitate and promote these reactions. The DME synthesis catalyst may be, for example, a copper-based catalyst, a copper oxide (CuO) based catalyst, a zinc oxide (ZnO) based catalyst, or a mixture of copper oxide and zinc oxide supported on alumina, or any combinations thereof. Other catalysts are applicable.

Conventional systems and processes generally have the sorbent mixed with the catalyst in the same bed. The mixed bed of catalyst and sorbent may be operated in cyclic mode, as fixed beds, subjected to process conditions favoring the main conversion reaction in one cycle, and at more severe conditions favoring the regeneration of sorbents typically at higher temperatures in the next cycle.

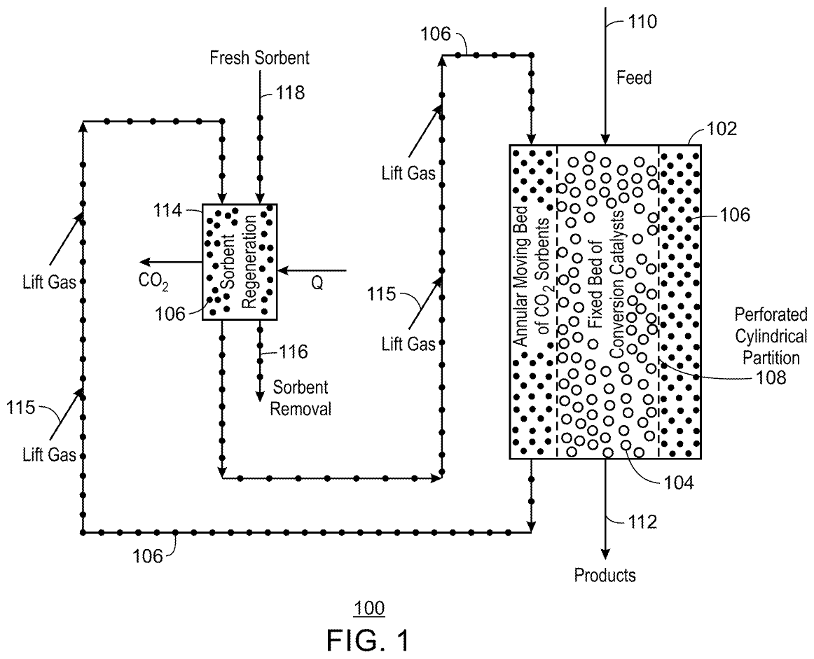

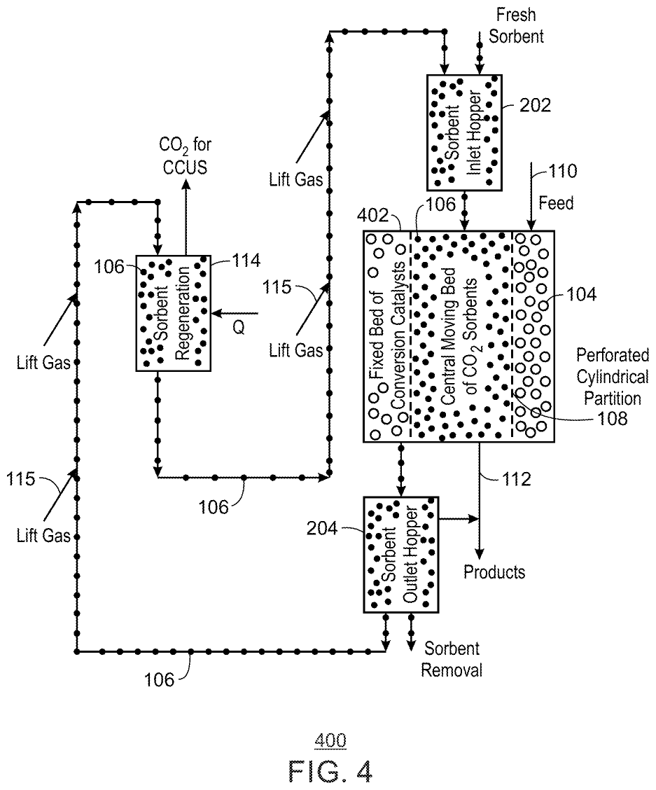

In contrast, embodiments of the present techniques (e.g., FIGS. 1-4) may facilitate selective removal (e.g., continuously) of CO₂ from the reaction environment of the reactor without subjecting the catalyst to the harsh conditions (e.g., temperature greater than reaction temperature) typically implemented for sorbent regeneration. This may be achieved by placing the catalyst within a fixed bed operating continuously in the reactor at the reaction conditions, and the sorbent in a moving packed bed continuously moving between the reaction and regeneration zones. In embodiments (e.g., FIG. 4), the moving packed bed may be located in the central core (radial center portion of the reactor) and the fixed catalyst bed located in the annular space around the radial center portion having the moving packed bed of sorbent. In other embodiments (e.g., FIGS. 1-3), the moving packed bed of sorbent may be located in the annular space around a core catalyst bed.

The two beds (fixed bed of catalyst and the moving bed or sorbent) may be separated, for example, by a perforated partition (e.g., perforated cylindrical partition) that facilitates movement (e.g., continuous movement) of reaction mixture inclusive of CO₂ from the catalyst bed to the sorbent bed for sorption of the CO₂ by the sorbent, thereby removing CO₂ from the reaction environment. The spent sorbent bed may be transported to a regenerator operating at elevated temperatures (e.g., temperatures greater than the operating temperature of the reactor), following which the regenerated sorbent may be returned to the sorbent section in the reactor. The hot regenerated sorbent returning to the reactor from the regenerator may beneficially provide heat for desired reactions in the catalytic reactor that are endothermic.

As indicated, this reactor-regenerator configuration can be applied to steam reforming of hydrocarbons (including methane), water-gas shift reaction, production of DME, and other equilibrium-limited reactions. CO₂ may become attached to the sorbent, e.g. limestone, dolomite, hydrotalcite or lithium-zirconate based sorbents. As an example, the following reactions may occur during the sorption and regeneration cycles with limestone as a sorbent:

Sorption (Carbonation): CaO+CO₂ [symbol] CaCO₃ ΔH₂₉₈⁰=−178 kJ/mol

Regeneration (Calcination): CaCO₃ [symbol] CaO+CO₂ ΔH₂₉₈⁰=178 kJ/mol.

The catalyst may be a solid as solid particles (catalyst particles). The sorbent may be a solid as solid particles (sorbent particles). The catalyst and sorbent can be, for example, pellets, granules, or coarse powder-having a particle size (e.g., diameter) greater than the hole or pore size (e.g., diameter) of the aforementioned perforated partition.

The catalytic reactors 102, 402 of FIGS. 1-4 may be cylindrical vessels having, for example, elliptical or semi-elliptical heads, and generally having a vertical orientation, though a horizontal orientation is not precluded. The reactor vessel has an outer wall and an inside volume (interior) internal with respect to the vessel wall. The reactor vessel may have nozzles, e.g., with flanged or screwed connections, for coupling to inlet and outlet conduits, and so forth. The inlet conduits may include a feed inlet conduit conveying reactants and a sorbent inlet conduit conveying regenerated sorbent. The outlet conduits may include an effluent conduit conveying reactor effluent (having product(s) and any unreacted reactants) discharged from the reactor vessel. The outlet conduits may include a sorbent outlet conduit conveying spent sorbent discharged from the reactor vessel (e.g., to the regenerator).

The reactor vessel may be a pressure vessel designed and configured (e.g., with adequate wall thickness) to be subjected to an internal pressure up to a specified pressure (design pressure) greater than ambient pressure (atmospheric pressure). A pressure vessel may be rated to hold a fluid up to the design pressure. In operation, the operating pressure in a pressure vessel may generally be maintained less than the design pressure. A pressure vessel may be constructed per a formal standard or code, such as the American Society of Mechanical Engineers (ASME) Boiler & Pressure Vessel Code (BPVC) or the European Union (EU) Pressure Equipment Directive (PED).

FIG. 1 is a reactor system 100 having a catalytic reactor 102 configured to perform an equilibrium-limited reaction that generates CO₂ as a product that is a byproduct. The reactor 102 includes a reactor vessel. The reactor 102 has a fixed bed of catalyst 104 in a radial center portion of the inside volume (interior) of the reactor vessel. This radial center portion may be labeled as a first region or center region of the inside volume. The first region and fixed bed of catalyst 104 therein are on (along and straddling) the longitudinal centerline of the reactor vessel. In operation, the catalyst 104 may promote the equilibrium-limited reaction(s) performed in the reactor vessel. In operation, the reactor 102 has a moving bed of sorbent 106 in an annular portion of the inside volume (interior) of the reactor vessel around the radial center portion having the fixed bed of catalyst 104. This annular portion may be labeled as a second region or annular region of the inside volume of the reactor vessel.

The first region (center portion region) having the catalyst 104 and the second region (annular region) having the sorbent 106 are separated by a perforated partition 108 (e.g., plate, screen, mesh, etc.) that is cylindrical in the reactor vessel. Thus, the first region and the second region as so separated may each be labeled as a chamber or a compartment. In the illustrated embodiment, the second region (annular region) is also bound by the outer wall of the reactor vessel. The perforated partition 108 may have pores or holes sized adequately small to prevent solids (e.g., catalyst 104 and sorbent 106) moving between the first region and the second region, but sized for passage of gas between the first region and the second region. The sorbent 106 and the catalyst 104 may have a particle size (e.g., diameter), for example, in range of 1 millimeter (mm) to 5 mm. In implementations, the size (e.g., diameter) of the holes in the perforated partition 108 may be, for example, less than 90% (or in a range of 50% to 90%) of the sorbent 106 particle size and/or the catalyst 104 particle size. The perforated partition 108 may restrict (prevent or substantially reduce) introduction of the catalyst 104 from the first region into the second region, and restrict (prevent or substantially reduce) introduction of sorbent 106 from the second region into the first region. Thus, the perforated partition 108 may restrict (prevent or substantially reduce) presence of the catalyst 104 from the first region in the second region and restrict (prevent or substantially reduce) presence of sorbent 106 from the second region in the first region. As discussed below, this reactor 102 configuration facilitates the sorbent 106 bed to be continuously regenerated in a cyclic manner without subjecting the catalyst 104 to stressful conditions (e.g., temperature greater than the reactor 102 operating temperature) of sorbent 106 regeneration that could accelerate deactivation of the catalyst 104 or otherwise degrade the catalyst 104.

This configuration may facilitate the catalyst 104 bed to operate continuously (without being interrupted for regenerating the sorbent 106) at process conditions (reaction conditions) beneficial for performance and longevity of the catalyst 104. Contemporaneously, the sorbent 108 bed may move downward due to gravity in the reactor 102 and discharged from the reactor 102, and be lifted up by a “lift” gas for vertical flow upward through a conduit to the regeneration section external of the reactor 102. The sorbent 106 can be regenerated in the regenerator (or regeneration zone) at a regeneration temperature greater than the reaction temperature of the reactor 102. Because the catalyst 104 is fixed in the reactor 102, the catalyst is beneficially not exposed to the typical greater temperature of regenerating the sorbent 106. The regenerated sorbent 106 bed maybe transferred from the regeneration zone by lift gas to the annular section (second region) of the reactor 102.

In operation, the reactor 102 receives a feed 110 including reactants giving a reaction mixture in the first region flowing through the fixed-bed of catalyst 104. The reaction mixture is the reactants fed to the reactor 102 plus products formed from the equilibrium-limited reaction of the reactants (promoted by the catalyst 104) in the reactor 102. The products may include at least a first product and CO₂. The reaction mixture may flow into the second region having the moving bed of sorbent 106. Thus, product CO₂ in the reaction mixture may sorb onto the sorbent 106, beneficially driving the equilibrium-limited reaction toward product formation. The reaction mixture may flow in both the first region and the second region. A reactor effluent 112 may discharge from the reactor 102 vessel, for instance, from the first region having the fixed-bed of catalyst 104. The reactor effluent 112 may have the products, plus any unreacted reactants remaining in the reaction mixture at the outlet of the reactor 102.

In operation, the sorbent 106 (as a moving bed) discharges from the reactor 102 vessel from the second region through an outlet conduit (e.g., tubing or piping) to a regeneration vessel 114. The regeneration vessel 114 is a sorbent regeneration vessel that in operation regenerates the sorbent 106. The combination of the discharge of sorbent 106 from the reactor 102 to the regeneration vessel 114 and the discharge of sorbent 106 from the regeneration vessel 114 to the reactor 102 may give a continuous cycle (recirculation, recycle) of the sorbent 106 (as a moving bed) in the system 100 with respect to the reactor 102.

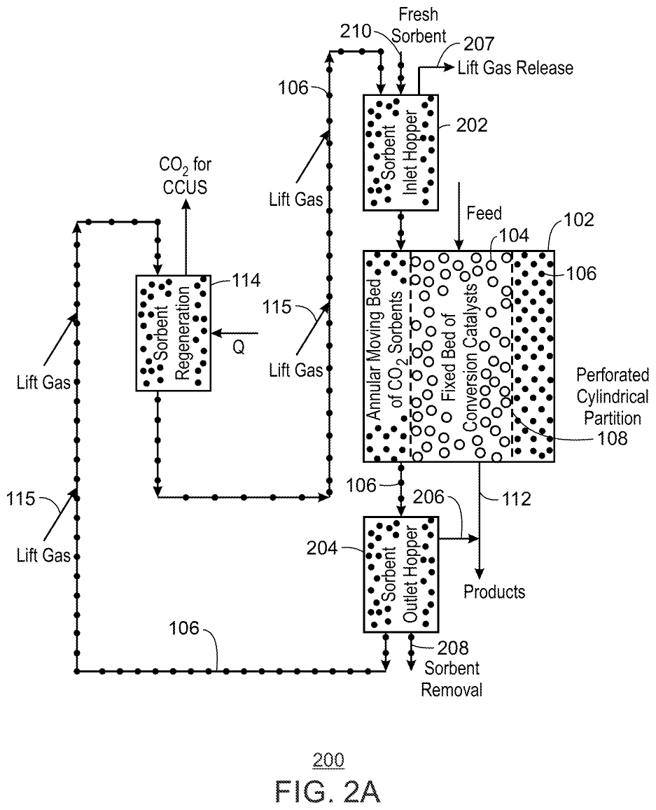

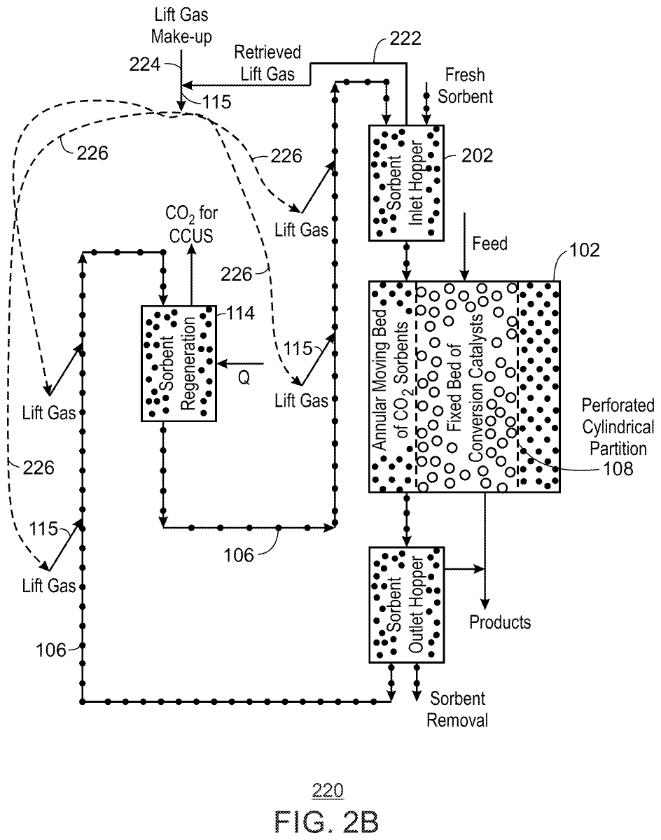

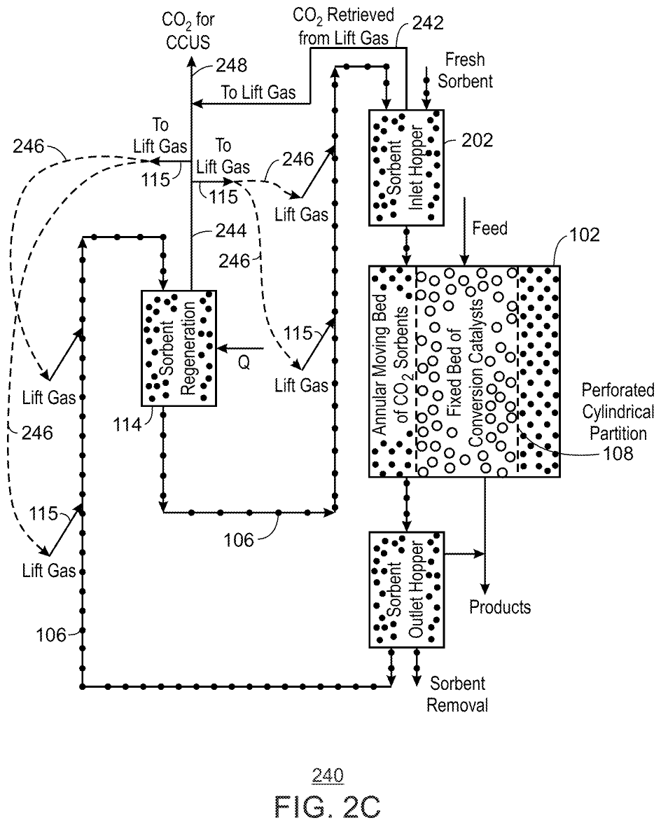

A lift gas 115 may be introduced in the circulation of the sorbent 106 and thus flow with the sorbent 106 in the outlet conduit to the regeneration vessel 114. Therefore, the flow through the outlet conduit may be two-phase flow including sorbent 106 particles and gas including lift gas 115. The lift gas 115 may be an inert gas (e.g., nitrogen gas) that is inert at least with respect to the reaction occurring in the reactor. The lift gas 115 can be CO₂, for instance, in implementations (e.g., FIG. 2C) discussed below in which lift gas is removed from the circulating moving bed of sorbent 106 such that little or no lift gas 115 flows through the reactor.

The lift gas 115 may be introduced into the circulating sorbent 106, such as from a common conduit header supply (e.g., a nitrogen [N₂] gas supply header) or from a tube bank of gas cylinders (for smaller scale operations). In implementations, a mechanical compressor may be employed to facilitate introduction of the lift gas 115 into the circulating sorbent 106 and promote flow of the lift gas 115 in the circulating sorbent 106. The lift gas 115 may be introduced, for example, into a conduit conveying the sorbent 106 between the reactor 102 and regeneration vessel 114. The lift gas 115 may be a conveying gas in facilitating and promoting flow of the sorbent 106 particles, including in both horizontal flow and vertical flow (e.g., through conduits). The lift gas 115 is labeled as “lift” gas because the lift gas 115 may be a primary driver of flow of the sorbent 106 particles in the vertical upward direction (in which the sorbent 106 is lifted up vertically). In contrast, in implementations, gravity can be a contributor for flow of the sorbent 106 particles in a vertical downward direction.

The sorbent 106 as discharged from the reactor 102 generally has sorbed CO₂ that is CO₂ sorbed from the reaction mixture in the reactor 102 vessel. The sorbent 106 as discharged having sorbed CO₂ may be labeled as “spent” sorbent because the discharged sorbent 106 in being used to sorb CO₂ has more sorbed CO₂ than the sorbent 106 that entered reactor 102. In implementations, the sorbent 106 as discharged from the reactor 102 can be saturated (or near saturation) in CO₂.

The sorbent 106 (e.g., spent sorbent) conveyed in the outlet conduit enters the regeneration vessel 114. Heat (Q) is added to regeneration vessel 114 to heat the sorbent 106 in the regeneration vessel 114 to desorb CO₂ from the sorbent 106 to regenerate the sorbent 106 to give the sorbent 106 as regenerated sorbent 106. The sorbent 106 as so regenerated has less sorbed CO₂ than the sorbent 106 that entered the regeneration vessel 114. The heating of the sorbent 106 to desorb CO₂ is regenerating the sorbent 106. The desorbed CO₂ may discharge from the regeneration vessel 114.

The moving bed operation of the sorbent 106 may facilitate removal 116 of sorbent from the circulation loop and addition 118 of fresh sorbent to the circulation loop. The replenishment rate of the sorbent may be estimated, for example, based on maintaining a beneficial level of sorption activity for the sorbent 106.

The CO₂ discharged from the sorbent regenerator (sorbent regeneration vessel 114) can be subjected to handling and/or processing, such as captured and sequestered, or purified and further utilized, and the like. As lift gas 115 may be flowing with the entering sorbent 106 into the regeneration vessel 114, some lift gas 115 may discharge with the CO₂ to the handling and/or processing. Moreover, some lift gas 115 may discharge with the sorbent 106 from the regeneration vessel 114 to the reactor 102. Further, lift gas 115 may be added (injected) to the sorbent 106 flowing from the regeneration vessel 114 to the reactor 102. In embodiments (e.g., FIGS. 2A-4), the lift gas 115 may separated from the sorbent 106 flowing from the regeneration vessel 114 to the reactor via an inlet hopper at the reactor before the sorbent 106 enters the reactor.

The heat (Q) may be added to the regeneration vessel 114, for example, by external heat provided through the walls of the regeneration vessel 114, such as via external electrical heaters mounted on the regeneration vessel 114, or via integrating (disposing) the regenerator vessel 114 in a furnace, or via external electromagnetic heaters applying electromagnetic form of heat, e.g., microwave heating, or any combinations thereof. The operating temperature of the regeneration vessel 114 may be, for example, in the range of 350° C. to 950° C., depending on the sorbent 106 employed. Example typical regeneration temperatures for desorbing CO₂ for different sorbents 106 are given in Table 1.

TABLE 1
Typical regeneration temperature of example sorbents

| | | Typical regeneration |
|---|---|---|
| | Sorbent | temperature (° C.) |
| Natural | Limestone (CaCO3) | 850-950 |
| sorbents | Dolomite/Huntite (CaCO3 + | 850-950 |
| | MgCO3) | |
| | Hydrotalcite | 350-450 |
| Synthetic | Lithium orthosilicate (Li4SiO4) | 700-800 |
| sorbents | Lithium zirconate (Li2ZrO3) | 650-750 |
| | Sodium zirconate (Na2ZrO3) | 750-850 |

The operating pressure in the regeneration vessel 114 may be, for example, in the range of 1 bar absolute to 35 bars absolute. The operating pressure can be outside this numerical range, as the operating pressure in the regeneration vessel 144 may generally depend on the operating conditions of the reactor 102 for the given equilibrium-limited reaction being performed in the reactor 102. The regeneration vessel 114 and the reactor 102 are coupled in the same flow loop, and therefore the pressure in the regeneration vessel 114 may depend on the pressure in the reactor 102 vessel. The pressure in the regeneration vessel 114 may be similar to the reactor 102 pressure, as adjusted for any pressure drop (fluid flow hydraulics) in sustaining the flow of the sorbent 108 solids between the reactor 102 vessel and the regeneration vessel 114. In implementations, the reactor 102 pressure can depend on downstream distribution or processing of the effluent 112. In other words, the reactor 102 pressure may be relied on to provide motive force for flow of the effluent 112 through an effluent discharge conduit from the reactor 102 to the downstream distribution or processing (and thus the downstream pressure can be a backpressure that drives the operating pressure of the reactor 102 in implementations). This may include if a mechanical compressor is not employed along the effluent discharge conduit. In applicable implementations, the reactor 102 pressure as motive force may overcome [1] the operating pressure of the downstream distribution or processing and [2] the pressure drop (due to frictional resistance) of effluent 112 flowing through the effluent discharge conduit to the downstream distribution or processing. For the example of a sorption-enhanced steam methane reforming (SE-SMR) system as the system 100, the steam methane reforming reaction in the reactor 102 may be thermodynamically favored at lower pressures, e.g., 1 or 2 bars absolute, but the reactor 102 may be operated commercially, for instance, between pressures of 25 to 30 bars absolute to provide motive force for flow of the effluent 112 through the effluent discharge conduit from the reactor 102 to the downstream systems.

The regenerated sorbent 106 discharges from the regeneration vessel 114 through an inlet conduit to the reactor 102 to give the moving bed of sorbent 106 moving through the second region (annular region) in the reactor 102 vessel. Lift gas may facilitate flow of the sorbent 106 through the inlet conduit to the reactor 102. In implementations, in the reactor 102, the sorbent 106 may move by gravity downward through the second [annular] region, though the lift gas can provide additional motive force for the downward flow. Yet, again, in implementations, the lift gas may be primarily for the movement of the sorbent 106 bed in the upward direction in the movement of the sorbent 106 in the system 100.

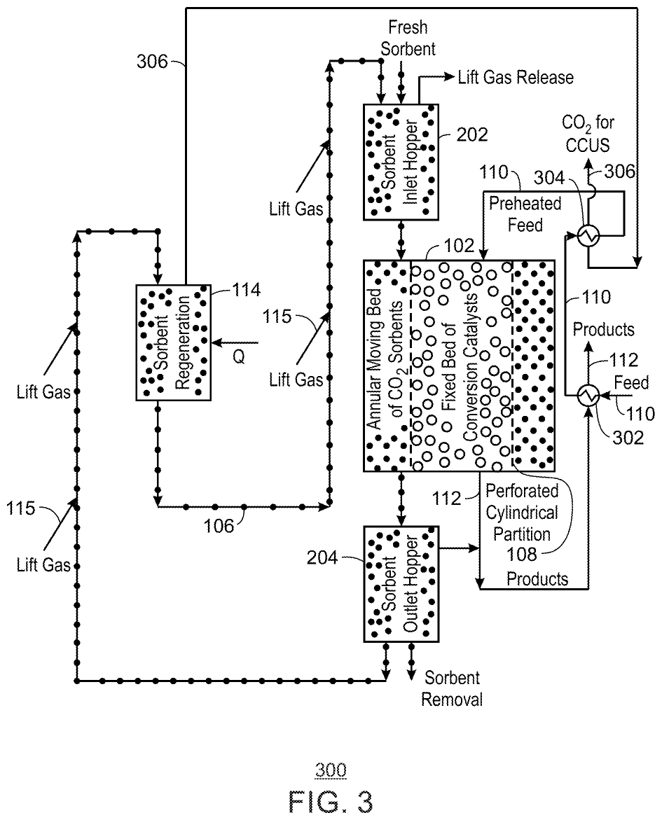

In implementations, the sorbent 106 discharged from the regeneration vessel 114 as regenerated and heated (e.g., the sorbent 106 having a temperature in the range of 350° C. to 950° C.) by the regeneration vessel 114 may provide heat for endothermic reactions in the reactor 102, thereby beneficially reducing the amount of aforementioned heating of the reactor 102. In the reactor 102, heat transfer may occur from the sorbent 106 to the reaction mixture. In certain implementations, the sorbent 106 as heated in the regeneration vessel 114 may provide adequate heat (sufficient) to sustain the endothermic equilibrium-limited reaction in the reactor 102. Therefore, in those implementations, the aforementioned heating (e.g., via external electrical heaters or other types of heating) of the reactor performing the endothermic equilibrium-limited reaction may not be needed nor employed. Moreover, in some implementations (e.g., FIG. 3), heat may be recovered from the hot CO₂ discharged from the regeneration vessel 114 (and heat recovered from the effluent 112) to heat the feed 110 to the reactor 102. In certain implementations, with heat provided via the regenerated sorbent 106 and whether or not the feed 110 is heated (preheated), some standard heating (e.g., via external electrical heaters) may be applied to reactor 102 to meet the reaction endothermic heat input and any heat losses.

The moving bed operation of the sorbent 106 may facilitate removal 116 of sorbent from the circulation loop and addition 118 of fresh sorbent to the circulation loop. In the illustrated embodiment, the removal 116 is depicted from the regeneration vessel 114, and the addition 118 is depicted to the regeneration vessel 114. The replenishment rate of the sorbent implemented may be estimated, for example, based on maintaining a beneficial level of sorption activity for the sorbent 106. The sorbent removal 116 and fresh sorbent addition 118 may be implemented to compensate for the loss in sorption capacity of the sorbent 106 over operation of several cycles of regeneration. For instance, if calcium carbonate (CaCO₃) is utilized as the sorbent 106, its sorption capacity may decrease in a particular example from 0.79 to 0.32 grams of CO₂ per gram of sorbent after 45 cycles of sorption/regeneration of the sorbent. In another particular case, the sorption capacity of dolomite sorbent decreases from 0.46 to 0.16 grams of CO₂ per gram of sorbent after 45 cycles.

As mentioned, in the reactor 102, the reaction mixture including products and feed (reactants) can flow from the first region having the catalyst 104 through the perforated partition 108 (through holes of the partition 108) to the second region having the sorbent 106. The product CO₂ from the reaction mixture (typically a reaction gas mixture) in the second region may be sorbed onto the sorbent 106. Some of the reaction mixture may discharge with the sorbent 106 through the outlet conduit to the regeneration vessel 114. As would be appreciated by one of ordinary skill in the art, the amount of reaction mixture (gas) flowing with the sorbent 106 can be limited through reactor design. Moreover, embodiments (e.g., FIGS. 2A-4) may employ a sorbent outlet hopper to remove and recover reaction mixture discharged with the sorbent 106 from the reactor 102. The sorbent 106 flowing to the regeneration vessel 114 can have a relatively small amount of reaction mixture (e.g., substantially CO₂-depleted). Thus, in examples of the reactor 102 as a steam methane reformer, relatively small amounts of unreacted methane gas, product H₂ gas, and product CO gas can flow with the sorbent 106 solid particles to the regeneration vessel 114. Indeed, some amount of the product gas mixture (CO₂-depleted) may be entrained with the spent sorbent 106 to the regeneration vessel 114. This will generally not cause adverse effect to the sorbent. The H₂ and any residual CH₄ and CO that goes along with the sorbent 106 and lift gas 115 to the regeneration vessel 114 may generate heat by combustion in the regeneration vessel 114. Thus, the reaction mixture flowing with the sorbent 106 and the lift gas 115 to the regenerator may (1) cause a corresponding loss of H₂ yield and (2) partially offset the energy required to regenerate the sorbent due to the combustion.

FIG. 2A is a reactor system 200 that may be analogous in configuration and operation to the reactor system 100 of FIG. 1, except that the reactor system 200 includes an inlet hopper 202 and an outlet hopper 204. The inlet hopper 202 is a vessel disposed along the inlet conduit to the catalytic reactor 102 from the regeneration vessel 114. The outlet hopper 204 is a vessel disposed along the outlet conduit from the reactor 102 to the regeneration vessel 114. The inlet hopper 202 and the outlet hopper 204 may more generally be a solids-gas separator in which solids settle (e.g., with aid of gravity) to the bottom portion of the hopper vessel for discharge of the solids from a bottom outlet of the hopper vessel. The gas that separates from the solids in the hopper vessel may discharge through an outlet on the upper portion of the hopper vessel. In certain implementations, the bottom portion of hopper vessel may have a conical shape extending narrowing toward the bottom of the hopper vessel. In some implementations, the hopper 202, 204 vessels may have internal baffles (e.g., impingement baffles) to disrupt gas flow and facilitate collection of the solids. Over the vertical longitudinal length of the hopper vessel, the solid particles may settle out of the gas toward the bottom. The hopper 202, 204 vessels may be configured in the system 200 with adequate residence time for the solid-gas separation to occur. In certain instances, operation of the hoppers 202, 204 can be more straightforward than other solids-gas separator vessel, such as a cyclonic separator vessel.

In operation, the sorbent 106 (as regenerated by the regeneration vessel 114) discharges from the regeneration vessel 114 and flows with lift gas (as two-phase flow of gas and solid) through the inlet conduit to the inlet hopper 202. The lift gas 115 may separate from the regenerated sorbent 106 via the inlet hopper 202. The regenerated sorbent 106 may discharge from a bottom portion of the inlet hopper 202 with little or no lift gas 115 to the catalytic reactor 102. The lift gas 115 may discharge 207 from an upper portion of the inlet hopper 202, such that no lift gas 115, or a relatively small amount of lift gas 115 (e.g., lift gas 115 entrained in the flow of sorbent 106), enters the reactor 102 from the inlet hopper 202. Thus, little or no lift gas 115 flows through the reactor 102.

The lift gas 115 discharged, as indicated by reference numeral 207, from the hopper 202 may be released as off-gas, such as to the environment (e.g., with the lift gas 115 as N₂) or to an off-gas collection system, and the like. Alternatively, if the lift gas 115 is an inert gas, e.g. N₂, the lift gas 115 can be recycled back to be utilized as lift gas. See, for example, FIG. 2B that depicts recycling lift gas within the system, with additional make-up feed of lift gas to account for any potential loss. In another alternative (e.g., FIG. 2C), if the lift gas is a split stream of CO₂ released from the sorbent regenerator 114, the lift gas 115 discharged from the inlet hopper 202 may either be released to the atmosphere or combined back with the CO₂ stream released from the regenerator 114. See, for example, FIG. 2C depicting utilizing a portion of CO₂ evolved from sorbent 106 in the regeneration vessel 114 as lift gas 115 in the system.

In operation as shown in FIG. 2A, the sorbent 106 (e.g., spent sorbent) as a moving bed discharges from the reactor 102 from the second [annular] region through the outlet conduit to the outlet hopper 204. The gas flowing with the sorbent 106 is separated in (via) the outlet hopper 204 and discharged as gas 206 from the outlet hopper to combine with the effluent 112 discharged from the reactor 102. The gas 206 may be reaction mixture that exited with the spent sorbent 106 from the reactor 102. In implementations, the gas 206 entering the outlet hopper 204 may be primarily the desired product(s) with any unreacted reactants, and may be substantially depleted in CO₂ in certain implementations due to sorption onto the sorbent 106. In embodiments, lift gas 115 may be introduced to the outlet conduit downstream of the outlet hopper 204 to facilitate any upward vertical movement of the spent sorbent 106 (moving bed) through the outlet conduit to the regeneration vessel 114. The CO₂ desorbed from the sorbent 106 in the regeneration vessel 114 may be provided from the regeneration vessel 114 for carbon, capture, utilization, and storage (CCUS).

Thus, in the illustrated embodiment of FIG. 2A, with the inlet hopper 202, the lift gas 115 may be separated and removed from the regenerated sorbent 106 entering back to the reactor 102. This can beneficially limit dilution of the reactants in the reactor. With the outlet hopper 204, gas (e.g., reaction mixture including product(s)) may be separated and removed from the spent sorbent 106 flowing to the regeneration vessel 114. This can recover any product from the sorbent 106 moving bed leaving the reactor 102 with the sorbent 106 stream. The sorbent outlet hopper 204 may facilitate separation of product gases entrained with the moving bed of sorbent 106 discharged from the reactor 102. As mentioned, the product gas or gases recovered via the sorbent outlet hopper 204 may be combined with the effluent 112 (having product) from the reactor 102 catalyst bed, thereby enhancing yield of the product(s) (e.g., valuable product such as H₂). Further, as mentioned, CO₂ from the sorbent regenerator (regeneration vessel 114) can be captured and sequestered, or purified and further utilized (CCUS).

As discussed with respect to FIG. 1, sorbent 106 in FIG. 2A may be removed from the system (from the circulating moving bed of sorbent), and fresh sorbent may be added to the system (to the circulating moving bed of sorbent). In the illustrated implementation of FIG. 2A, fresh sorbent 106 may be added 210 to the inlet hopper 202, and sorbent 106 may be removed 208 from the outlet hopper 204. As discussed, sorbent 106 may be removed and fresh sorbent added to compensate for the loss in sorption capacity of the sorbent 106 over operation of several cycles of regeneration.

FIG. 2B is a reactor system 220 that may generally be the reactor system 200 of FIG. 2A, but with the lift gas 115 (e.g., inert gas such as N₂) discharged from the inlet hopper 202 utilized as retrieved lift gas 115, as indicated by reference numeral 222. Lift gas make-up 224 may be added to the retrieved lift gas to give the lift gas 115 injected in the circulating sorbent 106 (moving bed) flowing through the outlet conduit from the reactor 102 to the regeneration vessel 114 and through the inlet conduit to the reactor 102 from the regeneration vessel 114. The dashed arrows 226 are representations of conduits (e.g., tubing or piping) that convey the lift gas 115 to the lift gas 115 injection points.

Thus, lift gas 115 discharged from the top portion of the inlet hopper 202 can be recycled back to be utilized as lift gas. In this recycling of lift gas 115 within the system 220, the additional make-up feed 224 of lift gas may account for losses of lift gas 115 from the system 220.

FIG. 2C is a reactor system 240 that may generally be the reactor system 200 of FIG. 2A, but with the lift gas 115 (e.g., CO₂) discharged from the inlet hopper 202 utilized as retrieved lift gas 115, as indicated by reference numeral 242. The retrieved lift gas may combined with CO₂ 244 discharged from the regeneration vessel 114 to be utilized as lift gas 115 for injection into the circulating sorbent 106 (moving bed). The dashed arrows 246 are representations of conduits (e.g., tubing or piping) that convey the lift gas 115 (retrieved lift gas plus CO₂ 244) conveyed to the lift gas 115 injection points. The arrow 248 is a representation of CO₂ from the regeneration vessel 114 not utilized as lift gas but sent, for example, to CCUS.

Thus, in FIG. 2C, the lift gas 115 may be a split stream (or slipstream) of CO₂ 244 released from the sorbent regenerator 114 combined with retrieved lift gas from the inlet hopper 202. Therefore, a portion of CO₂ evolved from sorbent 106 in the regeneration vessel 114 is lift gas 115 in the system 240.

FIG. 3 is a reactor system 300 that may be analogous in configuration and operation to the reactor system 100 of FIG. 1 and the reactor system 200 of FIG. 2A, having the inlet hopper 202 and the outlet hopper 204, but with FIG. 3 depicting the system 300 having a first heat exchanger 302 and a second heat exchanger 304 for heating the feed 110. Again, the feed 110 is generally the reactants fed to the catalytic reactor 102, which are reactants for the equilibrium-limited reaction performed in the reactor 102.

The first heat exchanger 302 and the second heat exchanger 304 may each be, for example, a shell-and-tube heat exchanger or a plate-fin type heat exchanger, and the like.

In operation, the first heat exchanger 302 heats the feed 110 with the effluent 112 from the reactor 102. The effluent 112 (as cooled by the feed 110 in the first heat exchanger 302) may discharge from the first exchanger 302 for downstream processing of the effluent 112. The second heat exchanger 304 heats the feed 110 with the CO₂ 306 discharged from the regeneration vessel 114. The CO₂ 306 may be CO₂ desorbed from sorbent 106 in the regeneration vessel 114. The CO₂ 306 (as cooled by the feed 110 in the second heat exchanger 304) may discharge from the second heat exchanger 304 to CCUS.

In the illustrated implementation, the feed 110 flows from the first heat exchanger 302 to the second heat exchanger 304. The feed 110 as heated by the first heat exchanger 302 and the second heat exchanger 304 discharges from the second heat exchanger 304 to the reactor 102. Thus, the feed 100 entering the reactor may be labeled as preheated feed. This preheating of the feed 110 combined with heat provided by the regenerated sorbent 106 entering the reactor provides heat for any endothermic reaction in the reactor 102, and thus may reduce energy and cost utilized in heating the reactor 102.

FIG. 4 is a reactor system 400 that may be analogous in configuration and operation to the reactor system 100 of FIG. 1 and the reactor system 200 of FIG. 2A, having the inlet hopper 202 and the outlet hopper 204, but with the catalytic reactor 402 having the moving bed of sorbent 106 in the first region and the fixed-bed of catalyst 104 in the second region.

Thus, the catalytic reactor 402 (for performing the equilibrium-limited reaction) may be analogous to the reactor 102 of the previous figures, but with the sorbent 106 (as a moving bed) in the radial center portion of the inside volume (interior) of the reactor vessel, labeled as the aforementioned first region or center portion region of the inside volume. This first region and moving bed of sorbent 106 therein may be on (along and straddling) the longitudinal centerline of the reactor vessel. As indicated, another distinction of the reactor 402 of FIG. 4 versus the reactor 102 of the previous figures is the fixed-bed of catalyst 104 is in an annular portion [the aforementioned second region] of the inside volume (interior) of the reactor vessel around the radial center portion [first region] having the moving bed of sorbent 106. This annular portion may be labeled as the aforementioned second region or annular region of the inside volume. As mentioned, the catalyst 104 may promote the equilibrium-limited reaction(s) performed in the reactor vessel. As discussed, the perforated partition 108 may restrict movement of catalyst 104 and sorbent 106 between the first region and the second region.

In operation, the sorbent 106 (moving bed) discharges from the first region from the reactor 402 through the outlet conduit (from the reactor 402) and the outlet hopper 204 disposed along the outlet conduit to the regeneration vessel 114. Lift gas 115 may be introduced to the outlet conduit between the outlet hopper 204 and the regeneration vessel 114 to promote (advance) flow of the sorbent 106 upward through vertical portion(s) of the outlet conduit. The effluent 112 (having at least a product of the equilibrium-limited reaction) discharges from the second region from the reactor 402, for example, to downstream distribution or processing.

The sorbent 106 (regenerated sorbent) discharges (e.g., as a moving bed) from the regeneration vessel 114 through the inlet conduit to the first region of the reactor 402. Lift gas 115 may be introduced to the inlet conduit between the regeneration vessel 114 and the inlet hopper 204 to promote (advance) flow of the sorbent 106 upward through vertical portion(s) of the inlet conduit. The regenerated sorbent 106 (with lift gas 115) flows through the inlet hopper 202 disposed along the inlet conduit. Lift gas 115 is removed from the sorbent 106 via the inlet hopper 202 before the sorbent 106 discharges from the inlet hopper 202 to the first region of the reactor 402.

The feed 110 having reactants for the equilibrium-limited reaction may be introduced to the reactor 402 to the second region. The reaction mixture having reactants and products of the equilibrium-limited reaction in the reactor 402 vessel may flow back and forth between the second region and the first region. The product CO₂ from the reaction mixture is sorbed onto the sorbent 106 (of the sorbent moving bed) in the first region. The reaction mixture depleted of the product CO₂ sorbed onto the sorbent 106 but having an additional product(s) of the equilibrium-limited reaction, any residual product CO₂, and any unreacted reactants, discharges as the effluent 112 from the reactor 402 from the second region in the illustrated implementation of FIG. 4.

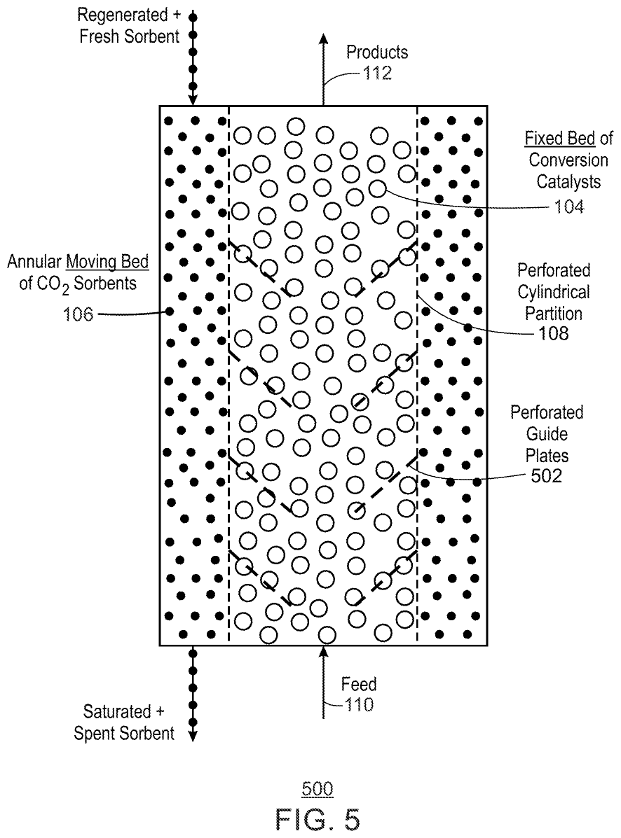

FIG. 5 is a catalytic reactor 500 that may be analogous to the reactors 102, 402 of the preceding figures, but guide plates 502 are depicted. Further, the feed 110 is introduced to a bottom portion of the reactor 500, and the effluent 112 discharges from a top portion of the reactor 500.

In the illustrated implementation, the fixed-bed of catalyst 104 is in the first region [central region] of the reactor 500. The moving bed of sorbent 106 moves downward through the second region [annular region] of the reactor 500. Regenerated sorbent 106 is introduced at the top portion of the reactor 500 into the second region. Spent sorbent 106 discharges from the bottom portion of the reactor from the second region. The perforated cylindrical partition 108 separates the first region from the second region.

In certain implementations, the guide plates 502 (e.g., perforated guide plates) may be relatively flat plates (e.g., metal) and extending across about a ⅓ (e.g., ¼ to ⅜) of the radial width of the catalyst 104 bed (and of the radial width of the first region). In operation, the guide plates 502 promote contact of the reaction mixture (reaction gas mixture) with the sorbent 106. In the illustrated example of FIG. 5, the feed 110 is introduced at the bottom of the catalyst 104 bed, with the reaction mixture flowing upwards, and products (effluent 112) received from the top of the central fixed bed of catalyst 104. The guide plates 502 may guide the reaction mixture through the annular moving bed (flowing downwards) of sorbent 106, guide the reaction mixture upwards through the holes in the guide plates 502 as perforated, and generally advance movement of the reaction mixture between the catalyst bed 104 and the moving bed of sorbent 106. This may advance sorption of the product CO₂ from the reaction mixture onto the sorbent 106. FIG. 5 depicts feed 110 upflow, with guide plates installed for gas-sorbent contact enhancement.

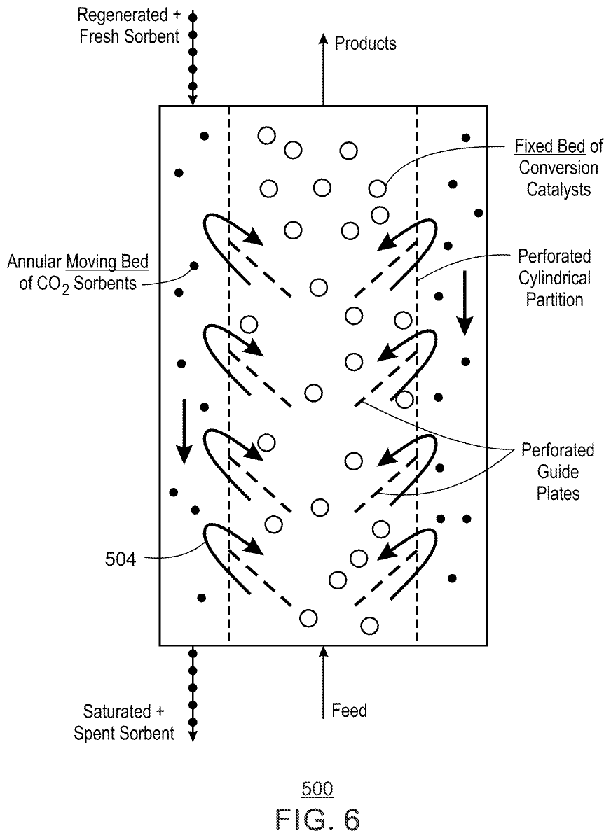

FIG. 6 is the catalytic reactor 500 of FIG. 5, but with some of the representations of catalyst 104 particles and sorbent 106 particles not depicted (removed) for clarity. FIG. 6 depicts expected gas path-lines 504 around the guide plates 502 that enhance gas-sorbent contact in increasing contact of the sorbent 106 with the reaction mixture, thereby increasing sorption of the product CO₂ from the reaction mixture onto the sorbent 106. Thus, the technique may include promoting flow of the reaction mixture between the first region and the second region via the guide plates 502 in the catalytic reactor 500. The technique may include altering flow of the reaction mixture via the guide plates 502 in the reactor vessel, wherein the reaction mixture flows in the first region and in the second region. Guide plates 502 installation is one configuration to enhance (increase) gas contact with the moving bed of sorbent 106. Other internal inserts can be designed and installed for the same or similar effect. Further, while the reactor 500 has the catalyst 104 in the first [central] region and the sorbent 106 in the second [annular] region, the utilization of guide plates 502 or other reactor internals to advance contact of the reaction mixture with the sorbent 106 can be applied in catalytic reactors having the sorbent 106 in the first [central] region and the catalyst 104 in the second [annular] region. Also, while the reactor 500 receives the feed 110 into the bottom portion of the reactor and discharges the effluent 112 from the top portion of the reactor 500, the utilization of guide plates 502 or other reactor internals to advance contact of the reaction mixture with the sorbent 106 can be applied in catalytic reactors that receive the feed 110 into the top portion of the reactor and discharge the effluent 112 from the bottom portion of the reactor.

The terms “first” and “second” with respect to the regions in the reactor vessel can be arbitrary. The discussion of FIGS. 1-6 has labeled the radial center portion of the inside volume of the reactor vessel as the first region and the annular portion of the inside volume radially around the first region as the second region. However, the radial center portion of the inside volume can be labeled as the second region and the annular portion of the inside volume radially around that second region labeled as the first region.

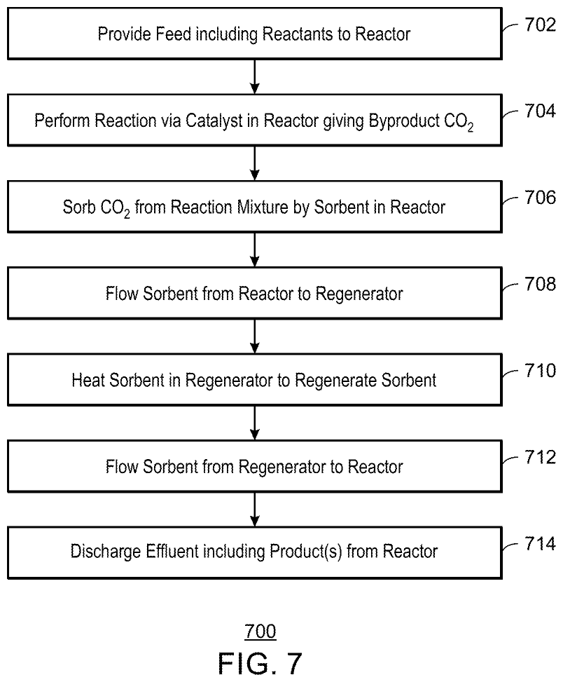

FIG. 7 is a method 700 of removing carbon dioxide from a reaction mixture in a reactor. The carbon dioxide in the reaction mixture may be a product (e.g., byproduct) of a reaction in the reactor. For embodiments with the reaction as an equilibrium-limited reaction, the removal of the product carbon dioxide may beneficially drive the equilibrium-limited reaction toward product formation.

At block 702, the method includes providing a feed including reactants to a reactor that is a catalytic reactor including a vessel (e.g., cylindrical vessel) that is a reactor vessel, wherein an inside volume (interior) of the vessel has a first region having a fixed bed of catalyst and a second region having a moving bed of sorbent separated from the first region by a perforated partition, such as a perforated cylindrical partition. In implementations, the perforated partition (e.g., plate, screen, mesh, etc.) restricts introduction of the catalyst from the first region into the second region and restricts introduction of sorbent from the second region into the first region. Thus, the perforated generally restricts movement of the catalyst and sorbent between the first region and the second region. In implementations, the perforated partition restricts presence of the catalyst from the first region in the second region and restricts presence of sorbent from the second region in the first region. In implementations, the catalyst does not contact the sorbent. In implementations, the catalyst is not in the moving bed of sorbent and the sorbent is not in the fixed bed of catalyst.

In implementations, the first region is a radial center portion of the inside volume of the vessel and the second region is an annular portion of the inside volume of the vessel radially around the first region. In other implementations, the second region is a radial center portion of the inside volume of the vessel and the first region is an annular portion of the inside volume of the vessel radially around the second region.

At block 704, the method includes performing in the reactor via the catalyst a reaction (e.g., an equilibrium-limited reaction) of the reactants giving products of the reaction, the products including a first product and carbon dioxide. In implementations, the carbon dioxide as a product is a byproduct.

At block 706, the method includes sorbing carbon dioxide in the reactor by the sorbent, thereby removing carbon dioxide from a reaction mixture having the reactants and the products in the reactor. The perforated partition may generally permit flow of the reaction mixture between the first region and the second region. The method may include promoting flow of the reaction mixture (e.g., through the perforated partition) between the first region and the second region via guide plates (or other reactor internals) in the reactor. The method may include altering flow of the reaction mixture via guide plates in the reactor vessel, wherein the reaction mixture flows in the first region and in the second region.

At block 708, the method includes moving (flowing) sorbent having sorbed carbon dioxide from the reactor via lift gas through an outlet conduit to a sorbent regeneration vessel. The method may include discharging the sorbent having sorbed carbon dioxide from the reactor vessel through the outlet conduit to a regeneration vessel. The discharging of the sorbent from the reactor vessel may include discharging the sorbent having sorbed carbon dioxide from the reactor vessel through an outlet hopper to remove gas (discharged from the reactor with the sorbent) flowing with the sorbent having sorbed carbon dioxide.

In implementations, the sorbent includes sorbent particles, wherein moving the sorbent having sorbed carbon dioxide from the reactor includes an outlet two-phase flow of gas and sorbent particles through the outlet conduit to the sorbent regeneration vessel.

As indicated, for example, in FIGS. 2A-4, this outlet two-phase flow may include a first two-phase flow and a second two-phase flow, the first two-phase flow through the outlet conduit between the reactor and an outlet hopper disposed along the outlet conduit, and the second two-phase flow through the outlet conduit between the outlet hopper and the sorbent regeneration vessel. The gas in the first two-phase flow includes the reaction mixture having the first product discharged from the reactor with the sorbent, and the gas in the second two-phase flow includes lift gas introduced into the outlet conduit between the outlet hopper and the sorbent regeneration vessel. The method may include removing the gas including the reaction mixture from the first two-phase flow via the outlet hopper and discharging the gas including the reaction mixture from the outlet hopper to combine with the effluent.

At block 710, the method includes heating the sorbent in the sorbent regeneration vessel to remove sorbed carbon dioxide giving regenerated sorbent having less sorbed carbon dioxide than the sorbent entering the sorbent regeneration vessel from the reactor. The method generally includes desorbing carbon dioxide from the sorbent in the regeneration vessel by heating the sorbent in the regeneration vessel. In implementations, the sorbent is not regenerated in the reactor, and wherein regenerating the sorbent via heating the sorbent in the sorbent regeneration vessel does not interrupt operation of the reactor.

At block 712, the method includes moving (flowing) the regenerated sorbent from the sorbent regeneration vessel via lift gas through an inlet conduit to the reactor as sorbent introduced to the reactor. The method may generally include discharging sorbent from the regeneration vessel without the carbon dioxide desorbed from the sorbent through the inlet conduit to the reactor vessel. The method may include discharging the sorbent from the regeneration vessel without the carbon dioxide desorbed from the sorbent through an inlet hopper disposed along the inlet conduit to remove lift gas flowing with the sorbent discharged from the regeneration vessel. In implementations, the sorbent includes sorbent particles, wherein moving the regenerated sorbent (as discharged) from the sorbent regeneration vessel includes an inlet two-phase flow of lift gas and sorbent particles through the inlet conduit toward the reactor. In implementations, the method includes flowing the inlet two-phase flow to the reactor through an inlet hopper vessel disposed along the inlet conduit, and removing lift gas from the inlet two-phase flow via the inlet hopper vessel. The sorbent may discharge from the bottom portion of the inlet hopper vessel (e.g., with aid of gravity) into the reactor vessel.

In implementations, moving (block 708) the sorbent having sorbed carbon dioxide from the reactor and moving (block 712) the regenerated sorbent to the reactor is a continuous operation giving the moving bed of sorbent in the reactor contemporaneous with (e.g., at the same time) introducing the feed to the reactor, contemporaneous with (e.g., at the same time) the reaction via the catalyst is occurring in the reactor, and contemporaneous with (e.g., at the same time) discharging (block 714) an effluent from the reactor.

At block 714, the method includes discharging the effluent having the products (at least one product) from the reactor. The effluent may include unreacted reactants.

The method may include heating the feed in a heat exchanger with the effluent or heating the feed in a heat exchanger with carbon dioxide discharged from the sorbent regeneration vessel. The method may include heating the feed in a first heat exchanger with the effluent and in a second heat exchanger with carbon dioxide discharged from the sorbent regeneration vessel.

In implementations, the reactor is a steam methane reformer, wherein the catalyst includes reforming catalyst, wherein the feed includes steam and natural gas having methane, wherein the reactants includes steam and methane, and wherein the products includes the first product that is hydrogen gas, a second product that is carbon monoxide, and the carbon dioxide. The natural gas may be primarily methane (e.g., 60 to 90 volume percent) and the balance including hydrocarbons (e.g., ethane, propane, etc.). The hydrocarbons other than methane may also be steam reformed. The natural gas may include acid gas (e.g., carbon dioxide and/or hydrogen sulfide) in certain instances.

In implementations, the reactor is a water-gas shift reactor, the catalyst includes shift catalyst, the reactants include carbon monoxide and water vapor, and the first product is hydrogen gas. In implementations, the reactor is a dimethyl ether (DME) synthesis reactor, the reactants include carbon monoxide and hydrogen gas, and the first product is DME.

As appreciated by one of ordinary skill in the art, an equilibrium-limited reaction is a reaction in which conversions of the reactants are limited by reaction equilibrium. Chemical reaction equilibrium may generally occur when the quantities of reactants and products in a reaction remain unchanged. In a chemical reaction, chemical equilibrium (e.g., a dynamic equilibrium) may be the state in which both the reactants and products are present in concentrations that have no further tendency to change with time. This state may result when the forward reaction proceeds at the same rate as the reverse reaction. The reaction rates of the forward and backward reactions are generally not zero, but they are equal. Thus, there are generally no net changes in the concentrations of the reactants and products. Therefore, the equilibrium by which a reaction may be equilibrium limited may be the reaction in equilibrium when the forward and reverse reactions occur at equal rates. Equilibrium does not necessarily mean that reactants and products are present in equal amounts, but means that the reaction has reached a point where the concentrations of the reactant and product are unchanging with time, because the forward and backward reactions have the same rate. Again, an equilibrium-limited reaction may be a reaction limited by the aforementioned equilibrium.

An embodiment is a method of removing carbon dioxide from a reaction mixture in a reactor, including providing a feed having reactants to a reactor that is a catalytic reactor including a vessel, wherein an inside volume of the vessel has a first region having a fixed bed of catalyst and a second region having a moving bed of sorbent separated from the first region by a perforated partition. In implementations, the perforated partition restricts introduction of the catalyst from the first region into the second region and restricts introduction of sorbent from the second region into the first region. In implementations, the catalyst does not contact the sorbent. In implementations, the vessel is generally a cylindrical vessel, wherein the perforated partition includes a perforated cylindrical partition, and wherein the first region is a radial center portion of the inside volume and the second region is an annular portion of the inside volume radially around the first region; or the second region is a radial center portion of the inside volume and the first region is an annular portion of the inside volume radially around the second region.

The method includes performing in the reactor via the catalyst a reaction (e.g., equilibrium-limited reaction) of the reactants giving products of the reaction, the products including a first product and carbon dioxide (e.g., as a byproduct). In implementations, the reactor is a steam methane reformer, wherein the catalyst includes reforming catalyst, wherein the feed includes steam and natural gas having methane, wherein the reactants include steam and methane, and wherein the products include the carbon dioxide, the first product that is hydrogen gas, and a second product that is carbon monoxide. In implementations, the reactor includes a water-gas shift reactor, the catalyst includes shift catalyst, the reactants include carbon monoxide and water vapor, and the first product is hydrogen gas. In implementations, the reactor includes a DME synthesis reactor, the reactants include carbon monoxide and hydrogen gas, and the first product is DME.

The method includes sorbing carbon dioxide in the reactor by the sorbent, thereby removing carbon dioxide from a reaction mixture having the reactants and the products in the reactor. In implementations, the perforated partition permits flow of the reaction mixture between the first region and the second region. The method may include promoting flow of the reaction mixture between the first region and the second region via guide plates in the reactor. The method includes moving sorbent having sorbed carbon dioxide from the reactor via lift gas through an outlet conduit to a sorbent regeneration vessel, heating the sorbent in the sorbent regeneration vessel to remove sorbed carbon dioxide giving regenerated sorbent having less sorbed carbon dioxide than the sorbent entering the sorbent regeneration vessel from the reactor, moving the regenerated sorbent from the sorbent regeneration vessel via lift gas through an inlet conduit to the reactor as sorbent introduced to the reactor, and discharging an effluent comprising at least one of the products (e.g., the first product) from the reactor. In implementations, heating the sorbent in the sorbent regeneration vessel to remove sorbed carbon dioxide involves desorbing carbon dioxide from the sorbent in the sorbent regeneration vessel. In implementations, the effluent includes unreacted reactant(s). The method may include heating the feed in a heat exchanger with the effluent, heating the feed in a heat exchanger with carbon dioxide discharged from the sorbent regeneration vessel, or heating the feed in a first heat exchanger with the effluent and in a second heat exchanger with carbon dioxide discharged from the sorbent regeneration vessel.

In implementations, moving sorbent having sorbed carbon dioxide from the reactor and moving regenerated sorbent to the reactor is a continuous operation giving the moving bed of sorbent in the reactor at same time the feed is provided to the reactor, the reaction via the catalyst is occurring in the reactor, and the effluent is discharged from the reactor, wherein the sorbent is not regenerated in the reactor, and wherein regenerating the sorbent via heating the sorbent in the sorbent regeneration vessel does not interrupt operation of the reactor. In implementations, the sorbent includes sorbent particles, wherein moving the sorbent having sorbed carbon dioxide from the reactor involves an outlet two-phase flow of gas and sorbent particles through the outlet conduit to the sorbent regeneration vessel, and wherein moving the regenerated sorbent from the sorbent regeneration vessel involves an inlet two-phase flow of lift gas and sorbent particles through the inlet conduit toward the reactor. The method may include flowing the inlet two-phase flow to the reactor through an inlet hopper vessel disposed along the inlet conduit, and removing lift gas from the inlet two-phase flow via the inlet hopper vessel. The outlet two-phase flow may involve a first two-phase flow and a second two-phase flow, the first two-phase flow through the outlet conduit between the reactor and an outlet hopper disposed along the outlet conduit, and the second two-phase flow through the outlet conduit between the outlet hopper and the sorbent regeneration vessel, wherein the gas in the first two-phase flow includes the reaction mixture having the first product discharged from the reactor with the sorbent, and wherein the gas in the second two-phase flow includes lift gas introduced into the outlet conduit between the outlet hopper and the sorbent regeneration vessel, and wherein the method may include removing the gas comprising the reaction mixture from the first two-phase flow via the outlet hopper and discharging the gas including the reaction mixture from the outlet hopper to combine with the effluent.

Another aspect relates to a method of removing carbon dioxide from a reaction mixture in a reactor, including providing a feed having reactants to a catalytic reactor including a reactor vessel, wherein an interior of the reactor vessel has a first region having a fixed bed of catalyst and a second region having a moving bed of sorbent, wherein the first region and the second region are separated by a perforated partition that restricts presence of the catalyst from the first region in the second region and restricts presence of sorbent from the second region in the first region. The method includes performing in the reactor vessel via the catalyst an equilibrium-limited reaction of the reactants giving products including carbon dioxide as a byproduct, and removing carbon dioxide from a reaction mixture including the reactants and the products in the reactor vessel by sorbing carbon dioxide from the reaction mixture onto the sorbent. The method includes discharging sorbent having sorbed carbon dioxide from the reactor vessel through an outlet conduit to a regeneration vessel, desorbing carbon dioxide from the sorbent in the regeneration vessel by heating the sorbent in the regeneration vessel, discharging sorbent from the regeneration vessel without the carbon dioxide desorbed from the sorbent through an inlet conduit to the reactor vessel, and discharging an effluent including the products from the reactor.

In implementations, the catalyst is not in the moving bed of sorbent, wherein the sorbent is not in the fixed bed of catalyst, and wherein the sorbent is not regenerated in the reactor vessel. In implementations, the method include altering flow of the reaction mixture via guide plates in the reactor vessel, wherein the reaction mixture flows in the first region and in the second region. In implementations, the discharging of the sorbent from the reactor vessel includes discharging the sorbent having sorbed carbon dioxide from the reactor vessel through an outlet hopper to remove gas discharged from the reactor with the sorbent and thus flowing with the sorbent having sorbed carbon dioxide. The discharging of the sorbent from the regeneration vessel may involve discharging the sorbent without the carbon dioxide desorbed from the sorbent through an inlet hopper disposed along the inlet conduit to remove lift gas flowing with the sorbent discharged from the regeneration vessel. The method may include heating the feed in a heat exchanger with the effluent, or heating the feed in a heat exchanger with carbon dioxide discharged from the regeneration vessel, or heating the feed in a first heat exchanger with the effluent and in a second heat exchanger with carbon dioxide discharged from the regeneration vessel. The reactor vessel may be a cylindrical vessel, wherein the perforated partition may include a perforated cylindrical partition, and wherein the first region is a radial center portion of the interior of the reactor vessel and the second region is an annular portion of the interior of the reactor vessel radially around the first region; or the second region is a radial center portion of the interior of the reactor vessel and the first region is an annular portion of the interior of the reactor vessel radially around the first region. In implementations, the reactor is a steam methane reformer, the reactants include steam and methane, and the products comprise hydrogen gas; or the reactor includes a water-gas shift reactor, the reactants include carbon monoxide and water vapor, and the products comprise hydrogen gas; or the reactor includes a DME synthesis reactor, the reactants include carbon monoxide and hydrogen gas, and the products include DME.

A number of implementations have been described. Nevertheless, it will be understood that various modifications may be made without departing from the spirit and scope of the disclosure.

What is claimed is:

1. A method of removing carbon dioxide from a reaction mixture in a reactor, comprising:
providing a feed comprising reactants to a reactor that is a catalytic reactor comprising a vessel, wherein an inside volume of the vessel comprises a first region having a fixed bed of catalyst and a second region having a moving bed of sorbent separated from the first region by a perforated partition;
performing in the reactor via the catalyst a reaction of the reactants giving products of the reaction, the products comprising a first product and carbon dioxide;
sorbing carbon dioxide in the reactor by the sorbent, thereby removing carbon dioxide from a reaction mixture comprising the reactants and the products in the reactor;
moving sorbent having sorbed carbon dioxide from the reactor via lift gas through an outlet conduit to a sorbent regeneration vessel;
heating the sorbent in the sorbent regeneration vessel to remove sorbed carbon dioxide giving regenerated sorbent having less sorbed carbon dioxide than the sorbent entering the sorbent regeneration vessel from the reactor;
moving the regenerated sorbent from the sorbent regeneration vessel via lift gas through an inlet conduit to the reactor as sorbent introduced to the reactor; and
discharging an effluent comprising at least one of the products from the reactor.

2. The method of claim 1, wherein the perforated partition restricts introduction of the catalyst from the first region into the second region and restricts introduction of sorbent from the second region into the first region, and wherein the perforated partition permits flow of the reaction mixture between the first region and the second region, and wherein the reaction is an equilibrium-limited reaction.

3. The method of claim 1, wherein the reactor is a steam methane reformer, wherein the catalyst comprises reforming catalyst, wherein the feed comprises steam and natural gas comprising methane, wherein the reactants comprise steam and methane, and wherein the products comprise the first product comprising hydrogen gas, a second product comprising carbon monoxide, and the carbon dioxide.

4. The method of claim 1, wherein:
the reactor comprises a water-gas shift reactor, the catalyst comprises shift catalyst, the reactants comprise carbon monoxide and water vapor, and the first product comprises hydrogen gas; or
the reactor comprises a dimethyl ether (DME) synthesis reactor, the reactants comprises carbon monoxide and hydrogen gas, and the first product comprises DME.

5. The method of claim 1, wherein the catalyst does not contact the sorbent, and wherein heating the sorbent in the sorbent regeneration vessel to remove sorbed carbon dioxide comprises desorbing carbon dioxide from the sorbent in the sorbent regeneration vessel.

6. The method of claim 1, wherein moving sorbent having sorbed carbon dioxide from the reactor and moving regenerated sorbent to the reactor is a continuous operation giving the moving bed of sorbent in the reactor at same time the feed is provided to the reactor, the reaction via the catalyst is occurring in the reactor, and the effluent is discharged from the reactor, wherein the sorbent is not regenerated in the reactor, and wherein regenerating the sorbent via heating the sorbent in the sorbent regeneration vessel does not interrupt operation of the reactor.

7. The method of claim 1, comprising promoting flow of the reaction mixture between the first region and the second region via guide plates in the reactor, wherein the carbon dioxide as a product is a byproduct.

8. The method of claim 1, wherein the sorbent comprises sorbent particles, wherein moving the sorbent having sorbed carbon dioxide from the reactor comprises an outlet two-phase flow of gas and sorbent particles through the outlet conduit to the sorbent regeneration vessel, and wherein moving the regenerated sorbent from the sorbent regeneration vessel comprises an inlet two-phase flow of lift gas and sorbent particles through the inlet conduit toward the reactor.

9. The method of claim 8, comprising flowing the inlet two-phase flow to the reactor through an inlet hopper vessel disposed along the inlet conduit, and removing lift gas from the inlet two-phase flow via the inlet hopper vessel, and wherein the effluent comprises unreacted reactants.

10. The method of claim 8, wherein the outlet two-phase flow comprises a first two-phase flow and a second two-phase flow, the first two-phase flow through the outlet conduit between the reactor and an outlet hopper disposed along the outlet conduit, and the second two-phase flow through the outlet conduit between the outlet hopper and the sorbent regeneration vessel, wherein the gas in the first two-phase flow comprises the reaction mixture comprising the first product discharged from the reactor with the sorbent, and wherein the gas in the second two-phase flow comprises lift gas introduced into the outlet conduit between the outlet hopper and the sorbent regeneration vessel, and wherein the method comprises removing the gas comprising the reaction mixture from the first two-phase flow via the outlet hopper and discharging the gas comprising the reaction mixture from the outlet hopper to combine with the effluent.

11. The method of claim 1, comprising heating the feed in a heat exchanger with the effluent, or heating the feed in a heat exchanger with carbon dioxide discharged from the sorbent regeneration vessel, or heating the feed in a first heat exchanger with the effluent and in a second heat exchanger with carbon dioxide discharged from the sorbent regeneration vessel.

12. The method of claim 1, wherein the vessel comprises a cylindrical vessel, wherein the perforated partition comprises a perforated cylindrical partition, and wherein:
the first region is a radial center portion of the inside volume and the second region is an annular portion of the inside volume radially around the first region; or
the second region is a radial center portion of the inside volume and the first region is an annular portion of the inside volume radially around the second region.

13. A method of removing carbon dioxide from a reaction mixture in a reactor, comprising:
providing a feed comprising reactants to a catalytic reactor comprising a reactor vessel, wherein an interior of the reactor vessel comprises a first region having a fixed bed of catalyst and a second region having a moving bed of sorbent, wherein the first region and the second region are separated by a perforated partition that restricts presence of the catalyst from the first region in the second region and restricts presence of sorbent from the second region in the first region;
performing in the reactor vessel via the catalyst an equilibrium-limited reaction of the reactants giving products comprising carbon dioxide as a byproduct;
removing carbon dioxide from a reaction mixture comprising the reactants and the products in the reactor vessel by sorbing carbon dioxide from the reaction mixture onto the sorbent;
discharging sorbent having sorbed carbon dioxide from the reactor vessel through an outlet conduit to a regeneration vessel;
desorbing carbon dioxide from the sorbent in the regeneration vessel by heating the sorbent in the regeneration vessel;
discharging sorbent from the regeneration vessel without the carbon dioxide desorbed from the sorbent through an inlet conduit to the reactor vessel; and
discharging an effluent comprising the products from the reactor.

14. The method of claim 13, wherein the catalyst is not in the moving bed of sorbent, wherein the sorbent is not in the fixed bed of catalyst, and wherein the sorbent is not regenerated in the reactor vessel.

15. The method of claim 13, comprising altering flow of the reaction mixture via guide plates in the reactor vessel, wherein the reaction mixture flows in the first region and in the second region.

16. The method of claim 13, wherein discharging the sorbent from the reactor vessel comprises discharging the sorbent having sorbed carbon dioxide from the reactor vessel through an outlet hopper to remove gas flowing with the sorbent having sorbed carbon dioxide, the gas discharged from the reactor with the sorbent.

17. The method of claim 13, wherein discharging the sorbent from the regeneration vessel comprises discharging the sorbent without the carbon dioxide desorbed from the sorbent through an inlet hopper disposed along the inlet conduit to remove lift gas flowing with the sorbent discharged from the regeneration vessel.

18. The method of claim 13, comprising heating the feed in a heat exchanger with the effluent, or heating the feed in a heat exchanger with carbon dioxide discharged from the regeneration vessel, or heating the feed in a first heat exchanger with the effluent and in a second heat exchanger with carbon dioxide discharged from the regeneration vessel.

19. The method of claim 13, wherein the reactor vessel comprises a cylindrical vessel, wherein the perforated partition comprises a perforated cylindrical partition, and wherein:
the first region is a radial center portion of the interior of the reactor vessel and the second region is an annular portion of the interior of the reactor vessel radially around the first region; or
the second region is a radial center portion of the interior of the reactor vessel and the first region is an annular portion of the interior of the reactor vessel radially around the first region.

20. The method of claim 13, wherein:
the reactor is a steam methane reformer, the reactants comprise steam and methane, and the products comprise hydrogen gas; or
the reactor comprises a water-gas shift reactor, the reactants comprise carbon monoxide and water vapor, and the products comprise hydrogen gas; or
the reactor comprises a dimethyl ether (DME) synthesis reactor, the reactants comprises carbon monoxide and hydrogen gas, and the products comprises DME.
